(19) United States
(12) Patent Application Publication
(10) Pub. No.: US 2026/0033983 A1
(43) Pub. Date: Feb. 5, 2026

(54) THERMAL THERAPY SYSTEM AND METHOD OF TEMPERATURE CONTROL THEREOF

(71) Applicant: DYNA INNOVATIONS INC., San Francisco, CA (US)

(72) Inventor: Omar CHATUR, Coquitlam (CA)

(21) Appl. No.: 18/998,402

(22) PCT Filed: Jul. 25, 2023
(86) PCT No.: PCT/IB2023/057545

§ 371 Date: Jan. 24, 2025

Related U.S. Application Data
(60) Provisional application No. 63/392,019, filed on Jul. 25, 2022.

(51) Int. Cl. A61F 7/02; A61F 7/00

(52) U.S. Cl. CPC A61F 7/02; A61F 7/007; A61F 2007/0075; A61F 2007/0078; A61F 2007/0086; A61F 2007/0093; A61F 2007/0096

(57) ABSTRACT

The present disclosure relates to a thermal therapy system for thermal medicine and/or therapy. The thermal therapy system comprises a thermal transfer device and a charging device. The thermal transfer device comprises a first portion made of a thermally conductive material and a second portion adapted to be fitted in a cavity of the first portion. where the first portion and the fitted second portion define the shape of the thermal transfer device. The thermal transfer device further comprises a temperature control element that is adapted to conform to the shape of the second portion. The temperature control element is disposed of within the cavity along with the second portion. Further, the actuation of the disposed temperature control element upon supplying electrical power of a predefined level adjusts the temperature of the thermal transfer device to a predefined temperature.

### CROSS-REFERENCE TO RELATED APPLICATION

This patent application claims the benefit of priority of U.S. Provisional Patent Application No. 63/392,019, filed on Jul. 25, 2022.

### FIELD

The present disclosure relates to the field of thermal therapy and systems therefor. More particularly, the present disclosure relates to a thermal therapy system and device for thermal therapy.

### BACKGROUND

Thermal therapies or thermotherapy is typically understood as the application of heat or cold (cryotherapy) for several treatment purposes, including changing the temperature of soft tissue for various treatments and regulating core body temperature for pain management, anxiety relief, performance and muscle recovery. In its most simplistic forms, thermal therapies include the application of a heat pack or a cold compress onto a desired location of the body to target specific muscles.

In one example, hot stone therapy is a well-established traditional treatment technique utilized for muscle relaxation, stress relief, and recovery. This therapeutic practice involves the application of smooth, heated stones on specific parts of the body to induce relaxation, alleviate muscle tension, and promote overall well-being. The stones, often made from basalt volcanic rock, may be heated to temperatures ranging from 50-60 degrees Celsius using different heating methods depending on the available technology and equipment.

Despite its popularity, the existing hot stone heating systems may have significant drawbacks that hinder the optimal delivery of this therapeutic procedure.

Firstly, it is limited to only the application of heat for treatments. Further, traditional heating methods, such as water-bath or “crockpot” heaters where stones are boiled in a large pot at approximately 140 degrees F. until the temperature for massage is achieved, have proven to be time-consuming, unsafe, and inconvenient for therapists and recipients. In addition, the process of boiling stones in a large pot may lead to accidents, inconsistent heating, and excessive humidity in the treatment room. Further, these stones may have to be dried before being used and they are often inconsistent in shape and size because of which they easily lose their heat.

Another alternative method involves heating plate or heating bag heaters, where the stones may be heated using a heating element, such as a heating plate or a flexible fabric with heating wires inside, however, this method may also suffer from delayed heating, uneven temperatures, and the use of suboptimal stone materials, which compromise treatment efficacy and overall customer satisfaction. Additionally, the risk of accidental burns when handling exposed conductive heating elements may also remain a concern. Due to these inherent limitations, many therapists may not choose to offer hot stone treatments.

Common ways for cryotherapy include the use of ice packs, cool whirlpool treatments (for example, ice baths), and freeze sprays, which can contain volatile propellants, such as pentane and butane. The use of ice packs, topical cooling agents, and freeze sprays are limited in their applications because it is difficult to regulate the temperature applied to the skin with the use of an ice pack, or freeze sprays. Further, such methods can only be used for very short periods of time, and the temperature applied cannot be consistent inbetween applications. The use of a cooling whirlpool of an ice bath requires substantial equipment, which may not be readily accessible to the average practitioner of thermal therapies.

There is a need for a system that enables anyone to easily access safe, effective thermal therapies for improved health and wellness. There is a need for a system that provides effective pain management, autonomic modification for reduce anxiety, improved athletic performance and faster muscle recovery.

### SUMMARY

The present disclosure relates to a simple, safe, efficient, and easy-to-use thermal therapy system for thermal therapy, which includes a thermal transfer device with an in-built temperature element and a charging device.

Described herein is a thermal transfer device with an in-built temperature element and a charging device for the thermal transfer device. The thermal transfer device comprises a first portion made of a thermally conductive material and a second portion adapted to be fitted in a cavity of the first portion, wherein the first portion and the fitted second portion define a shape of the thermal transfer device; and a temperature control element that is adapted to conform to a shape of the second portion, wherein the temperature control element is disposed of within the cavity along with the second portion, wherein actuation of the disposed temperature control element upon supplying electrical power of a predefined level, adjusts temperature of the thermal transfer device to a predefined temperature.

In one or more embodiments, the temperature control element is a heating element.

In one or more embodiments, the temperature control element is a thermoelectric element that is configured to cool and/or heat the thermal transfer device based on the predefined level of electrical power being supplied to the thermoelectric element.

In one or more embodiments, the second portion is wedge-shaped.

In one or more embodiments, the cavity is at the base of the first portion, wherein the base of the first portion is fluidically sealed once the temperature control element is disposed of within the cavity along with the second portion.

In one or more embodiments, the thermal transfer device comprises a temperature sensor configured at a tip of the second portion, such that when the second portion is fitted in the first portion along with the temperature control element, the temperature sensor remains at a center of the thermal transfer device to monitor temperature of the thermal transfer device from inside.

In one or more embodiments, the thermal transfer device comprises a set of electrical traces having a first end electrically connected to the temperature control element and/or the temperature sensor, wherein a second end of the set of electrical traces is connected to a circuit board comprising electrical pins that remain accessible or extend at least partially outside of the thermal transfer device once the temperature control element is fitted within the first portion along with the second portion.

In one or more embodiments, the thermal transfer device comprises a trim panel having openings fitted over a base of the cavity such that the electrical pins remain accessible or extend at least partially outside of the thermal transfer device through the openings of the trim panel.

In one or more embodiments, the second portion is press-fitted into the cavity of the first portion using a thermal paste applied on the second portion prior to the insertion into the cavity.

In one or more embodiments, an outer surface of the thermal transfer device is coated with a durable and non-porous material.

In one or more embodiments, the thermal therapy system comprises a charging device adapted to be electrically and operatively coupled to the thermal transfer device, wherein the charging device comprises a charging circuit that is configured to enable supply of electrical power of the predefined level from an in-built battery and/or an external power source to the thermal transfer device once the thermal transfer device is connected to the charging device.

In one or more embodiments, the charging device comprises a controller operatively coupled to the battery and configured to be operatively coupled to the thermal transfer device once the thermal transfer device is connected to the charging device, wherein the controller comprises one or more processors coupled to a memory storing instructions executable by the processors and configured to monitor, using the temperature sensor, real-time temperature of the thermal transfer device, allow one or more users to set the predefined temperature for the thermal transfer device, and based on the monitored real-time temperature and the predefined temperature, enable the supply of electrical power of the predefined level from the battery and/or the external power source to the temperature control element to adjust the temperature of the thermal transfer device to the predefined temperature.

In one or more embodiments, the charging device comprises a communication unit operatively coupled to the controller and configured to communicably couple the charging device to one or more mobile devices associated with the one or more users.

In one or more embodiments, the charging device comprises a housing defining a shape of the charging device, wherein the housing comprises at least one slot adapted to accommodate the thermal transfer device, such that upon accommodation, the thermal transfer device gets operatively coupled to the controller and the battery.

In one or more embodiments, the charging device is form of a handle that is removably or fixedly coupled to the thermal transfer device, wherein a housing of the handle is made of a thermally insulative material.

In one or more embodiments, the thermal transfer device or the charging device comprises an accelerometer to monitor kinematic attributes of the thermal transfer device, wherein the kinematic attributes comprise linear and angular speed, and orientation of the thermal transfer device; and a pressure sensor to monitor pressure applied by the one or more users while using the thermal transfer device.

In one or more embodiments, the thermal transfer device or the charging device comprises a haptic feedback device configured to provide haptic feedback to the one or more users holding the handle for controlling the movement of the thermal transfer device based on any or a combination of a predefined therapeutic treatment procedure selected by the one or more users using the mobile devices, the monitored kinematic attributes, and the monitored pressure applied by the one or more users in real-time.

In one or more embodiments, the charging device comprises a heat sink and a fan to facilitate dissipation of heat from the charging device and/or the thermal transfer device to facilitate adjusting the temperature of the thermal transfer device to the predefined temperature.

In one or more embodiments, the controller is configured to switch the thermal transfer device between an idle state and an active state, wherein the controller switches the thermal transfer device to the idle state when the controller detects the thermal transfer device, using an accelerometer, to be in a stationary position for a predefined time, and wherein the controller switches the thermal transfer device to the active state when the controller detects, using the accelerometer, the movement of the thermal transfer device.

In one or more embodiments, the thermal transfer device or the charging device comprises a LED strip configured on an outer surface of the thermal transfer device or charging device; and/or a display unit to display one or more of a charging status of the thermal transfer device, the predefined temperature set by the one or more users, and monitored real-time temperature of the thermal transfer device.

Various objects, features, aspects and advantages of the present disclosure will become more apparent from the following detailed description of preferred embodiments, along with the accompanying drawing figures in which like numerals represent like features.

Within the scope of this application, it is expressly envisaged that the various aspects, embodiments, examples and alternatives set out in the preceding paragraphs, in the claims and/or in the following description and drawings, and in particular the individual features thereof, may be taken independently or in any combination. Features described in connection with one embodiment are applicable to all embodiments unless such features are incompatible.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure. The diagrams are for illustration only, which thus is not a limitation of the present disclosure.

FIGS. 1A to 1C illustrate exemplary cross-sectional views of the thermal transfer device with built-in temperature control element, in accordance with one or more embodiments of the present disclosure;

FIG. 1D illustrates an exemplary perspective exploded view of the thermal transfer device, in accordance with one or more embodiments of the present disclosure;

FIG. 2 illustrates an exemplary exploded view of the charging device for the thermal transfer device, in accordance with one or more embodiments of the present disclosure.;

FIG. 3 illustrates a block diagram of the charging device of FIG. 2, in accordance with one or more embodiments of the present disclosure;

FIGS. 4A and 4B illustrates exemplary views of the thermal transfer device accommodated on the charging device, in accordance with one or more embodiments of the present disclosure;

FIGS. 5A to 5F illustrates an exemplary front view, right-side view, top view, bottom view, left-side view, and rear view, respectively, of a second embodiment of the thermal transfer device having a handle, in accordance with one or more embodiments of the present disclosure;

FIGS. 6A and 6B illustrates exemplary views of a cooling system equipped with a charging device for removing heat buildup from the thermal transfer device, in accordance with one or more embodiments of the present disclosure; and

FIG. 7 illustrates an exemplary network architecture of the thermal therapy system, where the thermal transfer device and the charging device are monitorable as well as controllable via mobile devices of users, in accordance with one or more embodiments of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

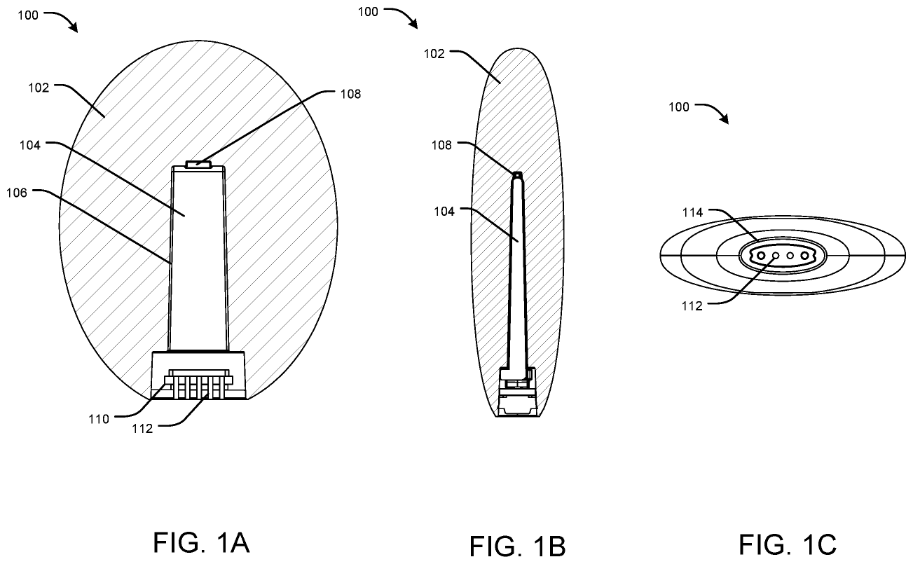

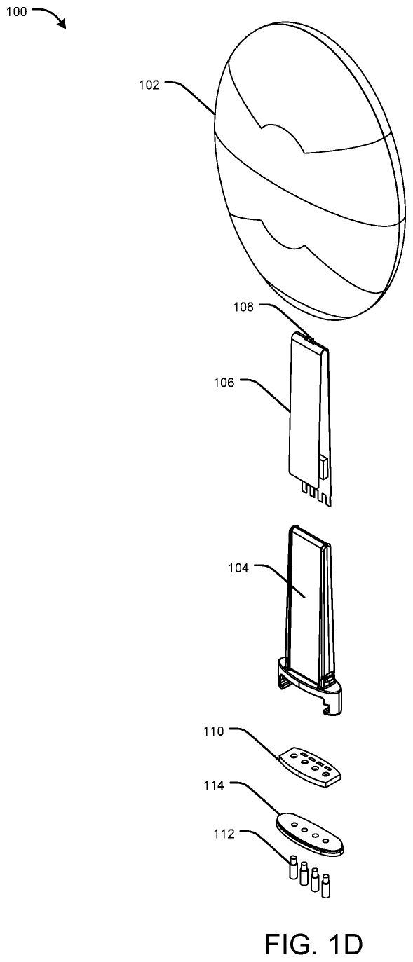

Referring to FIGS. 1A to 1D, the proposed thermal therapy system can include a thermal transfer device 100 with a built-in temperature control mechanism. The thermal transfer device 100 can include a first portion 102 defining the majority shape of the thermal transfer device 100, and a second portion 104 that is adapted to be fitted in a cavity of the first portion 102 to define the complete shape of the thermal transfer device 100. In an embodiment, a majority of the thermal transfer device 100 can be cast from a single piece of the first portion 102 with the cavity at the bottom or base of the first portion 102 to allow easier fitting of the second portion 104 in the first portion 102, however, the cavity can also be provided at other positions in the first portion 102 without any limitation.

In one or more embodiments, the second portion 104 can be wedge-shaped, but is not limited to the like, and the cavity can also have a profile based on the shape of the second portion 104 to provide a tight fitting between the second portion 104 and the first portion 102. While the thermal transfer device 100 is shown has having an oval shape or profile, embodiments of the invention need not be limited to this particular shape, and the thermal transfer device 100 can be made to be of any shape, including but not limited to egg shape, oval shape, pebble shape, disc shape, spherical shape, and disc shape with curved upper and lower surfaces, and other shapes appropriate for thermal therapy. In one or more embodiments, both the first portion 102 and the second portion 104 can be made of a thermally conductive but electrically insulative material. However, the first portion 102 and the second portion 104 need not be made of the same material.

The thermal transfer device 100 can further include a temperature control element 106 that can be adapted to conform to the shape of the second portion 104. The temperature control element 106 can be disposed of within the cavity along with the second portion 104, such that the second portion 104 can retain the temperature control element 106 within the first portion 102. In an implementation, the actuation of the disposed temperature control element 106, upon supplying electrical power of a predefined level, can accordingly adjust the temperature of the thermal transfer device 100 to a predefined temperature.

In one or more embodiments, the temperature control element 106 can be a heating element 106 made of a flexible electrically conductive material of high resistivity. The heat generated by the heating element 106 can be based on the voltage supplied, the resistance of the heating element 106, the resistance of electrical traces connected, and the electrical power supplied to the heating element 106. In other embodiments, the temperature control element 106 can be a thermoelectric element that is configured to cool and/or heat the thermal transfer device 100.

The thermal transfer device 100 can further include a temperature sensor 108 positioned at the tip of the second portion 104, such that when the second portion 104 is fitted in the first portion 102 along with the temperature control element 106, the temperature sensor 108 remains at the center or core of the thermal transfer device 100 to monitor the temperature of the thermal transfer device 100 from inside. In one or more embodiments, the temperature sensor 108 can be a surface mount, embedded temperature sensor 108, which can be placed at the tip of the wedge-shaped second portion 104.

The thermal transfer device 100 can include a set of electrical traces having a first end electrically connected to the temperature control element 106 and the temperature sensor 108. Further, the second end of the set of electrical traces can be connected to a printed circuit board 110 including one or more electrical pins 112 that remain accessible or extend at least partially outside of the thermal transfer device 100 once the temperature control element 106 is fitted within the first portion 102 along with the second portion 104. The electrical pins 112 can facilitate the establishment of an electrical connection between the heating element 106/temperature sensor 108 and a charging device 200 (or external power source). In addition, the thermal transfer device 100 can include a trim panel 114 having openings, being fitted over the base such that the electrical pins 112 remain accessible or extend at least partially outside of the thermal transfer device 100 through the openings of the trim panel 114. In an embodiment, the trim panel 114 can be made of plastic or silicon rubber, but not limited to the like, which can have a shape based on the profile of the base of the cavity.

In one or more embodiments, the second portion 104 can be press-fitted into the cavity of the first portion 102 using a thermal paste being applied on the second portion 104 before the insertion into the cavity, such that no fasteners or screws are required. In an implementation, once the circuit board 110 (PCB), is soldered and connected to the electrical traces connecting the temperature control element 106 and the integrated temperature sensor 108, the second portion 104 can be press-fitted into the cavity of the first portion 102. The base of the thermal transfer device 100 can then be sealed with a permanent adhesive material such as potting compound, and further fitted with the trim panel 114 that fits tightly around the pins such that after curing, the entire thermal transfer device 100 is formed into a single integrated and watertight unit.

Further, the thermal transfer device 100 can be coated with a durable, non-porous coating using techniques involving but not limited to powder-coating or physical vapor deposition (PVD), such that the thermal transfer device 100 can provide an optimal interaction surface with human skin at temperatures. Moreover, the non-porous coating may provide better hygienic, and bacterial and viral resistance. Further, the non-porous coating also avoids the absorption of oils used during therapy, thereby making it easier to clean the thermal transfer device 100.

Further, in other embodiments, the temperature control element 106 can be a thermoelectric element 106 that can be configured to cool and/or heat the thermal transfer device 100 based on the predefined level of electrical power being supplied to the thermoelectric element 106.

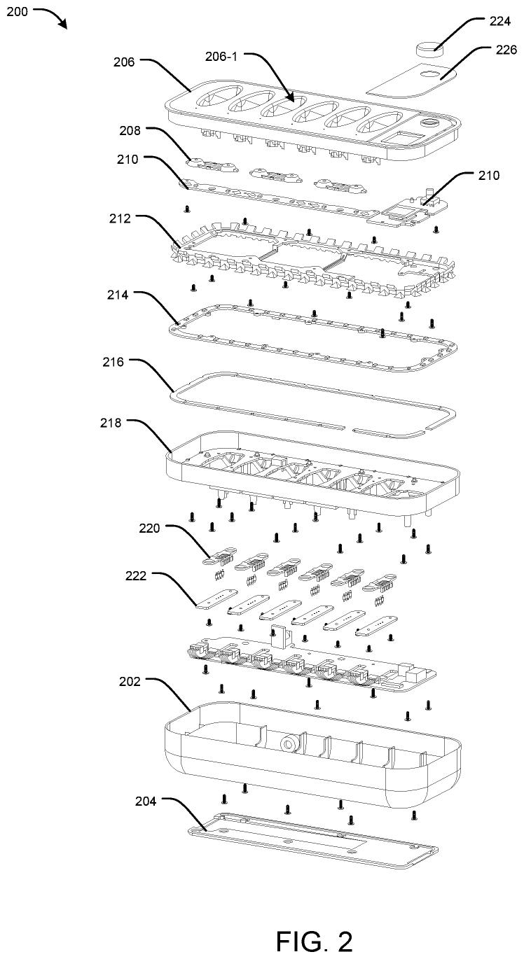

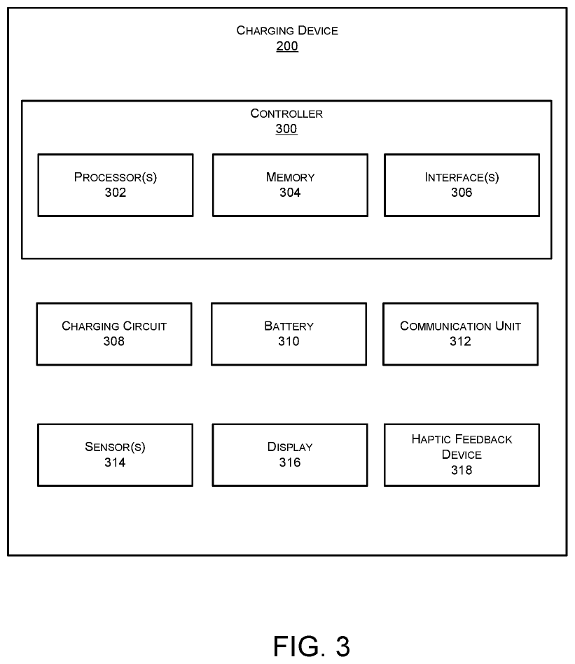

Referring to FIGS. 2 and 3, the thermal therapy system can further include a charging device 200 adapted to be electrically and operatively coupled to the thermal transfer device 100. The charging device 200 can include a charging circuit 308 that can be configured to enable the supply of electrical power of the predefined level from an in-built battery 310 and/or an external power source to the thermal transfer device 100 once the thermal transfer device 100 is connected to the charging device 200. Further, the charging device 200 can include a controller 300 operatively coupled to the battery. The controller 300 gets operatively coupled to the thermal transfer device 100 once the thermal transfer device 100 is connected to the charging device 200.

In an embodiment, the charging circuit 308 can include a power amplifier that can be configured to receive electrical power from the battery 310 or external power source and further convert and supply electrical power at the predefined level to the thermal transfer device 100 based on the power requirements of the thermal transfer device 100 and the predefined temperature selected by the users. In addition, the charging circuit 308 can include an analog-to-digital converter (ADC) configured to convert AC power received from the external power source to a DC power source for the battery 310 and/or the components of the charging device 200.

In one or more embodiments, the controller 300 can include one or more processors 302 coupled to a memory 304 storing instructions executable by the processors 302 and configured to enable the controller 300 or charging device 200 to perform one or more designated operations. The controller 300 can be configured to monitor, using the temperature sensor 108, real-time temperature of the thermal transfer device 100, and also allow one or more users to set the predefined temperature for the thermal transfer device 100. Accordingly, based on the monitored real-time temperature and the predefined temperature set by the users, the controller 300 or charging device 200 can enable the supply of electrical power (of the predefined level) from the battery and/or the external power source to the temperature control element 106 to adjust the temperature of the thermal transfer device 100 to the predefined temperature.

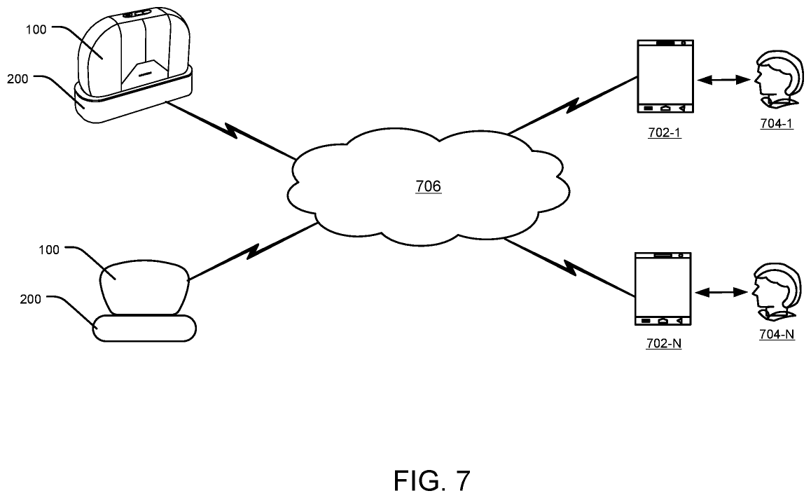

In one or more embodiments, the charging device 200 can include a communication unit or a transceiver 312 operatively coupled to the controller 300. Referring to FIG. 7, the communication unit 312 can be configured to communicably couple the charging device 200 to one or more mobile devices 702-1, 702-2 (individually referred to as mobile device and collectively referred to as mobile devices 702, herein) associated with the users 704-1, 704-2 (individually referred to as user and collectively referred to as users 704, herein). The users 704 may be therapists or normal individuals. Accordingly, the users 704 may remotely monitor and control the operation of the thermal transfer device 100 and the charging device 200 using their mobile devices 702.

The thermal therapy system can be operatively coupled to a remote server and so be operable from any internet-enabled mobile device 702. Examples of mobile devices 702 may include but are not limited to, a smartphone, a portable computer, a personal digital assistant, a handheld device, and a workstation. The mobile device 702 can be communicatively coupled or in communication with the thermal transfer device 100 or charging device 200 through the network 706.

In one or more implementations, the network 706 can be a wireless network, a wired network or a combination thereof. Network 706 can be implemented as one of the different types of networks, such as intranet, local area network (LAN), wide area network (WAN), the internet, and the like. Further, the network 706 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further, network 706 can include a variety of network devices, including transceivers, routers, bridges, servers, computing devices, storage devices, and the like. The network 706 can be a cellular network or mobile communication network based on various technologies, including but not limited to, Global System for Mobile (GSM), General Packet Radio Service (GPRS), Code Division Multiple Access (CDMA), Long Term Evolution (LTE), WiMAX, 5G or 6G network protocols, and the like.

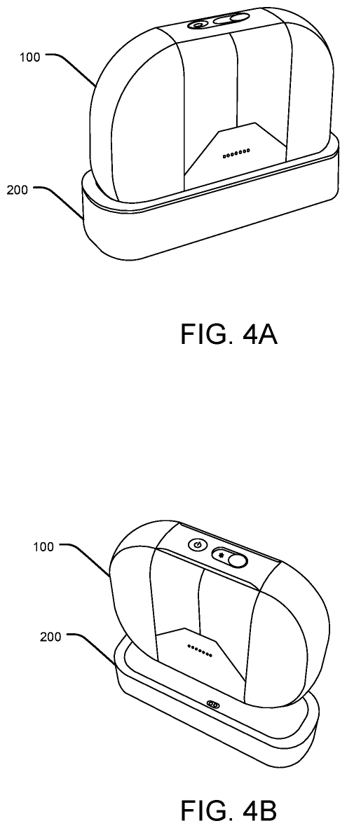

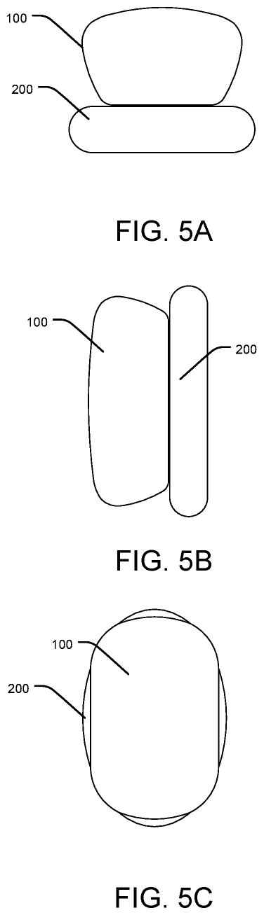

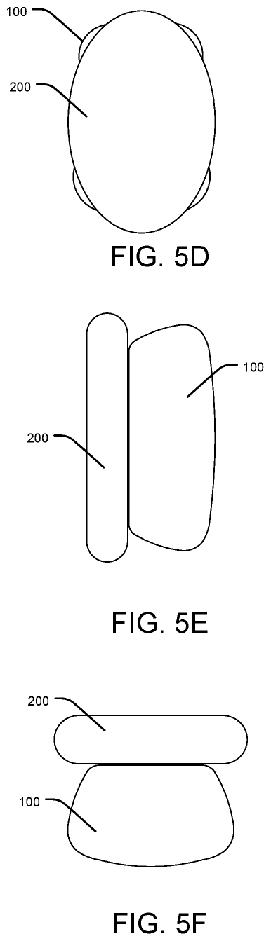

Referring back to FIGS. 2 and 3, in one or more embodiments, the charging device 200 can include a housing defining the shape of the charging device 200. The housing can include a bottom base 202 having side walls 218 and a base plate 204. Further, a top cover 206 can be fitted on the bottom base 202 using a gasket or sealing element 216 to define the shape of the housing and seal the interior components of the charging device 200. The housing can further securely accommodate the battery 310 therein. Further, the housing or top cover 206 of the charging device 200 can include at least one slot 206-1 adapted to receive and accommodate a number of the massage devices 100 therein at a time, as illustrated in FIGS. 4A to 5F, such that upon accommodation, the corresponding thermal transfer device 100 gets operatively coupled to the controller 300 and the battery associated with the charging device 200.

The charging device 200 can further include LED lights 208 provided at each of the slots 206-1, which can be further connected to different circuit boards and switches 210 for individually displaying the charging status of the respective thermal transfer device 100. Further, the charging device 200 can include a support mount 212 to support the circuit boards 210 and the LED lights 208 in the housing, and a mounting bracket 214 for the components of the charging device 200. In addition, the charging device 200 can include one or more charging contacts 220 provided in each of the slots 206-1, such that the charging contacts 220 can get electrically connected to the electrical pins 112 of the thermal transfer device 100 once the thermal transfer device 100 is accommodated in the slots of the charging device 200. These charging contacts 220 can further be electrically connected to the controller 300 and the battery 310 of the charging device 200 via different circuit boards (collectively referred to as the charging circuit 308, herein). The controller 300 or the charging circuit 308 can accordingly control the supply of electrical power from the battery 310 to the temperature control element 106 and temperature sensor 108 of the thermal transfer device 100, once accommodated/placed in the slots 206-1 of the charging device 200, to adjust the temperature of the thermal transfer device 100 at the predefined temperature. Further, the charging device 200 can include one or more support mounts (or mount brackets) 222 to support the charging contacts 220 and the associated circuit boards in the housing. Furthermore, the charging device 200 can include a switch 224 configured on the top cover via a cover plate 226, which allows users to turn ON and turn OFF the charging device 200. Furthermore, the housing can include a side cavity (not shown) to allow easier removal of any fluid dripped from the outer surface of the massage device 100 placed in the slots 206-1 of the charging device 200.

In one or more embodiments, the thermal transfer device 100 and/or charging device 200 can include a LED strip or light 208 configured on its outer surface. The LED 208 may provide an indication pertaining to the real-time charging status and the temperature of the thermal transfer device 100. Further, the thermal transfer device 100 and/or charging device 200 can also include a display unit 316 to display the charging status of the thermal transfer device 100 as well as the predefined temperature set by the users and the real-time temperature of the thermal transfer device 100.

In one or more embodiments, the charging circuit 308 can enable the charging device 200 to be electrically connected to an external power source, which may allow charging of the battery 308 associated with the charging device 200 and also allow direct transfer of electrical power from the external power source to the thermal transfer device 100 accommodated in the slots of the charging device 200. The housing may include another slot comprising a power port that facilitates establishing an electrical connection between the charging device 200 and the external power source.

Referring to FIGS. 5A to 5F, the charging device 200 can be in the form of a handle that can be configured to be removably or fixedly coupled to the thermal transfer device 100. The housing of the handle can be made of a thermally insulative material, which may restrict the transfer of heat or cold from the thermal transfer device 100 to the charging device 200/handle, thereby allowing the users to safely hold and use the thermal transfer device 100 without any injury. The handle 200 can accommodate the battery 310 and the charging circuit 208 which may allow charging of the battery and also allow direct transfer of electrical power from the external power source to the massage device 100.

In one or more embodiments, the handle/charging device 200 can include an accelerometer to monitor the kinematic attributes of the thermal transfer device 100, including but not limited to linear and angular speed, and orientation of the thermal transfer device 100. The handle can further include a pressure sensor to monitor pressure applied by the users while using the thermal transfer device 100. The accelerometer and the pressure sensor are collectively designated as 314 in FIG. 3. Further, the handle/charging device 200 can also include a haptic feedback device 318 to provide haptic feedback to the users 704 while holding the handle/charging device 200. The haptic feedback may facilitate the users 704 in controlling the movement of the thermal transfer device 100 based on any or a combination of a predefined thermal treatment and/or therapy selected by the users 704 using the mobile devices 702, the monitored kinematic attributes, and the monitored pressure applied by the users 704 in real time. In other embodiments, the sensors (accelerometer and pressure sensor) 314, and the haptic feedback device 318 can also be directly installed within the first portion 102 of the thermal transfer device 100, and all such embodiments are also well within the scope of this invention.

In an implementation, the controller 300 may be configured with one or more thermal therapies or treatment procedures, which can be installed as a set of instructions in a database of the controller 300. The thermal therapy system or controller 300 may allow the users 704 to select any of the therapies or treatment procedures using their mobile devices 702. Accordingly, the controller 300 may actuate the temperature control element 106 to adjust the temperature of the thermal transfer device 100 as per the selected therapy and display the steps for the selected therapy or procedure on the mobile device 702 of the users 704. The controller 300 may display the target muscle area, pressure required, type of motion required (if any), and duration for application of the thermal transfer device 100 on the recipient. In addition, the controller 300 may monitor the kinematic attributes and pressure applied by the users 704 on the recipient in real time and accordingly adjust the actuation of the haptic feedback device and the temperature of the thermal transfer device 100 as per the selected therapy or treatment procedure. The controller 300 may actuate the haptic feedback device to provide feedback regarding speeding up or slowing down the (gliding) motion of the thermal transfer device 100 on the recipient.

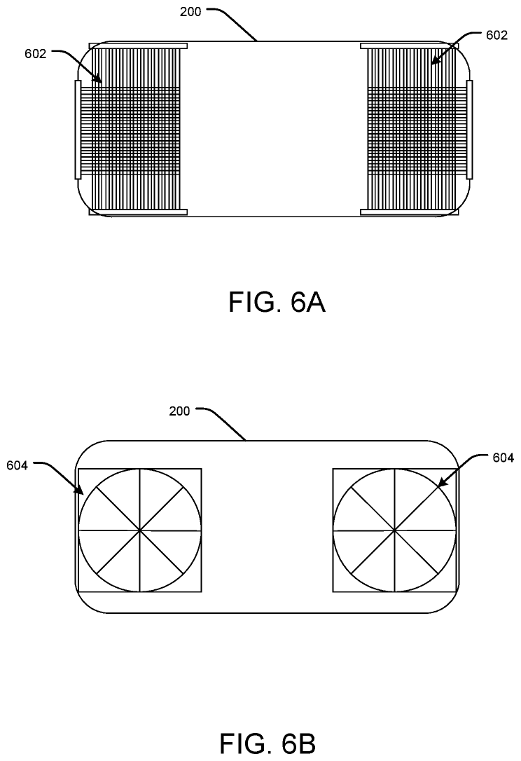

Further, referring to FIGS. 6A and 6B, in one or more embodiments, the charging device 200 can include a heat sink 602 in thermal contact with the housing of the charging device 200 and one or more DC fans 604 to facilitate quick dissipation of heat from the charging device 200 and/or the thermal transfer device 100, thereby quickly controlling and adjusting the temperature of the thermal transfer device 100 to the predefined temperature. However, the charging device 200 may include other types of cooling systems as well without any limitation.

In one or more embodiments, the controller 300 can be configured to switch the thermal transfer device 100 between an idle state and an active (wake) state. The controller 300 can switch the thermal transfer device 100 to the idle state when the controller 300 detects the thermal transfer device 100, using the accelerometer, to be in a stationary position for a predefined time. Further, the controller 300 can switch the thermal transfer device 100 to the active state when the controller 300 detects, using the accelerometer, the movement or lifting of the thermal transfer device 100 by the users. This may help in saving the battery of the charging device 200, thereby making the thermal therapy system power efficient.

Thus, the present disclosure overcomes the above-mentioned drawbacks, shortcomings, and limitations associated with existing heat stone therapy systems and methods, by providing a simple, safe, efficient, and easy-to-use system for thermal therapy, which includes a thermal transfer device with an in-built temperature control element and a charging device. In addition, as the temperature control element heats and/or cools the thermal transfer device from its core, the thermal transfer device gets evenly heated or cooled around its outer surface. Moreover, the temperature sensor keeps monitoring the temperature of the thermal transfer device from inside and the controller accordingly keeps adjusting the actuation of the temperature control element to maintain the temperature of the thermal transfer device at the same user-selected temperature, thereby preventing overheating/underheating or cooling/overcooling of the thermal transfer device. Further, the thermal therapy massage system allows users to remotely monitor and control the operation of the thermal transfer device. The thermal transfer device further allows users to select various thermal therapy procedures and accordingly provides feedback to the users to perform the selected therapy.

Referring back to FIG. 3, the controller 300 can include one or more processor(s) 302 operatively coupled to a memory 304. The one or more processor(s) 302 may be implemented as one or more microprocessors, microcomputers, microcontroller 300s, digital signal processors, central processing units, logic circuitries, and/or any devices that manipulate data based on operational instructions. Among other capabilities, processor(s) are configured to fetch and execute computer-readable instructions stored in the memory of the controller 300. The memory 304 may store one or more computer-readable instructions or routines, which may be fetched and executed to create or share the data units over a network service. The memory 304 may comprise any non-transitory storage device including, for example, volatile memory such as RAM, or non-volatile memory such as EPROM, flash memory, and the like.

The controller 300 also comprises an interface(s) 306 that may comprise a variety of interfaces, for example, interfaces for data input and output devices, referred to as I/O devices, storage devices, and the like. The interface(s) 306 includes the communication module to facilitate communication of the thermal therapy system with the mobile device, through the network. The interface(s) may also provide a communication pathway for one or more internal components or units of the controller 300 and with the mobile device. Examples of such internal components include, but are not limited to, processing engine(s) and data (database).

The processing engine(s) is implemented as a combination of hardware and programming (for example, programmable instructions) to implement one or more functionalities of the processing engine(s). In the examples described herein, such combinations of hardware and programming may be implemented in several different ways. For example, the programming for the processing engine(s) may be processor-executable instructions stored on a non-transitory machine-readable storage medium, and the hardware for the processing engine(s) may comprise a processing resource (for example, one or more processors), to execute such instructions. In the present examples, the machine-readable storage medium may store instructions that, when executed by the processing resource, implement the processing engine(s). In such examples, the controller 300 comprises the machine-readable storage medium storing the instructions and the processing resource to execute the instructions, or the machine-readable storage medium may be separate but accessible to the controller 300 and the processing resource. In other examples, the processing engine(s) may be implemented by an electronic circuitry. The database may comprise data that is either stored or generated as a result of functionalities implemented by any of the components of the processing engine(s) or thermal therapy system. The database may also store the one or more thermal therapy procedures.

It would be obvious to a person skilled in the art that while various embodiments and drawings of the present disclosure elaborate upon the first portion and second portion of the thermal transfer device having specific shapes for the sake of easier explanation, however, the teachings of the present disclosure are equally applicable for other shapes as well, and all such embodiments are well within the scope of the present disclosure without any limitation.

While some embodiments of the present disclosure have been illustrated and described, those are completely exemplary in nature. The disclosure is not limited to the embodiments as elaborated herein only and it would be apparent to those skilled in the art that numerous modifications besides those already described are possible without departing from the inventive concepts herein. All such modifications, changes, variations, substitutions, and equivalents are completely within the scope of the present disclosure. The inventive subject matter, therefore, is not to be restricted except in the spirit of the appended claims.

1. A thermal therapy system comprising:
a thermal transfer device comprising:
a first portion made of a thermally conductive material and a second portion adapted to be fitted in a cavity of the first portion, wherein the first portion and the fitted second portion define a shape of the thermal transfer device; and
a temperature control element that is adapted to conform to a shape of the second portion, wherein the temperature control element is disposed of within the cavity along with the second portion, wherein actuation of the disposed temperature control element upon supplying electrical power of a predefined level, adjusts temperature of the thermal transfer device to a predefined temperature.

2. The thermal therapy system of claim 1, wherein the temperature control element is a heating element.

3. The thermal therapy system of claim 1, wherein the temperature control element is a thermoelectric element that is configured to cool and/or heat the thermal transfer device based on the predefined level of electrical power being supplied to the thermoelectric element.

4. The thermal therapy system of claim 1, wherein the second portion is wedge-shaped.

5. The thermal therapy system of claim 1, wherein the cavity is at a base of the first portion, wherein the base of the first portion is fluidically sealed once the temperature control element is disposed of within the cavity along with the second portion.

6. The thermal therapy system of claim 1, wherein the thermal transfer device further comprises a temperature sensor configured at a tip of the second portion, such that when the second portion is fitted in the first portion along with the temperature control element, the temperature sensor remains at a center of the thermal transfer device to monitor temperature of the thermal transfer device from inside.

7. The thermal therapy system of claim 6, wherein the thermal transfer device further comprises a set of electrical traces having a first end electrically connected to the temperature control element and/or the temperature sensor, wherein a second end of the set of electrical traces is connected to a circuit board comprising electrical pins that remain accessible or extend at least partially outside of the thermal transfer device once the temperature control element is fitted within the first portion along with the second portion.

8. The thermal therapy system of claim 7, wherein the thermal transfer device further comprises a trim panel having openings fitted over a base of the cavity such that the electrical pins remain accessible or extend at least partially outside of the thermal transfer device through the openings of the trim panel.

9. The thermal therapy system of claim 1, wherein the second portion is press-fitted into the cavity of the first portion using a thermal paste applied on the second portion prior to the insertion into the cavity.

10. The thermal therapy system of claim 1, wherein an outer surface of the thermal transfer device is coated with a durable and non-porous material.

11. The thermal therapy system of claim 1, wherein the thermal therapy system comprises a charging device that is adapted to be electrically and operatively coupled to the thermal transfer device,
wherein the charging device comprises a charging circuit that is configured to enable supply of electrical power of the predefined level from an in-built battery and/or an external power source to the thermal transfer device once the thermal transfer device is connected to the charging device.

12. The thermal therapy system of claim 11, wherein the charging device comprises:
a controller operatively coupled to the battery and configured to be operatively coupled to the thermal transfer device once the thermal transfer device is connected to the charging device, wherein the controller comprises one or more processors coupled to a memory storing instructions executable by the processors and configured to:
monitor, using the temperature sensor, real-time temperature of the thermal transfer device;
allow one or more users to set the predefined temperature for the thermal transfer device; and
based on the monitored real-time temperature and the predefined temperature, enable the supply of electrical power of the predefined level from the battery and/or the external power source to the temperature control element to adjust the temperature of the thermal transfer device to the predefined temperature.

13. The thermal therapy system of claim 12, wherein the charging device comprises a communication unit operatively coupled to the controller and configured to communicably couple the charging device to one or more mobile devices associated with the one or more users.

14. The thermal therapy system of claim 11, wherein the charging device comprises a housing defining a shape of the charging device, wherein the housing comprises at least one slot adapted to accommodate the thermal transfer device, such that upon accommodation, the thermal transfer device gets operatively coupled to the controller and the battery.

15. The thermal therapy system of claim 11, wherein the charging device is in form of a handle that is removably or fixedly coupled to the thermal transfer device, wherein a housing of the handle is made of a thermally insulative material.

16. The thermal therapy system of claim 15, wherein the thermal transfer device or the charging device comprises:
an accelerometer to monitor kinematic attributes of the thermal transfer device, wherein the kinematic attributes comprise linear and angular speed, and orientation of the thermal transfer device; and
a pressure sensor to monitor pressure applied by the one or more users while using the thermal transfer device.

17. The thermal therapy system of claim 16, wherein the thermal transfer device or the charging device comprises;
a haptic feedback device configured to provide haptic feedback to the one or more users holding the handle for controlling the movement of the thermal transfer device based on any or a combination of a predefined treatment procedure selected by the one or more users using the mobile devices, the monitored kinematic attributes, and the monitored pressure applied by the one or more users in real-time.

18. The thermal therapy system of claim 11, wherein the charging device comprises one or more heat sinks and one or more fans to facilitate dissipation of heat from the charging device and/or the thermal transfer device to facilitate in adjusting the temperature of the thermal transfer device to the predefined temperature.

19. The thermal therapy system of claim 12, wherein the controller is configured to switch the thermal transfer device between an idle state and an active state,
wherein the controller switches the thermal transfer device to the idle state when the controller detects the thermal transfer device, using an accelerometer, to be in a stationary position for a predefined time, and
wherein the controller switches the thermal transfer device to the active state when the controller detects, using the accelerometer, the movement of the thermal transfer device.

20. The thermal therapy system of claim 11, wherein the thermal transfer device or the charging device comprises:
a LED strip or light configured on an outer surface of the thermal transfer device or charging device; and/or
a display unit to display one or more of charging status of the thermal transfer device, the predefined temperature set by the one or more users, and monitored real-time temperature of the thermal transfer device.
